(19) United States
(12) Patent Application Publication
(10) Pub. No.: US 2013/0195794 A1
(43) Pub. Date: Aug. 1, 2013

(54) CANCER VACCINE

(71) Applicants: Andrew Heath, Sheffield (GB); Jennifer Carlring-Wright, Sheffield (GB)

(72) Inventors: Andrew Heath, Sheffield (GB); Jennifer Carlring-Wright, Sheffield (GB)

(73) Assignee: Adjuvantix Limited

(21) Appl. No.: 13/581,113

(22) PCT Filed: Feb. 25, 2011
(86) PCT No.: PCT/GB2011/050372

§ 371 Date: Feb. 26, 2013

(30) Foreign Application Priority Data
Feb. 26, 2010 (GB) 1003293.6

(51) Int. Cl. A61K 39/00

(52) U.S. Cl. CPC A61K 39/0011
USPC 424/85.2; 424/178.1; 424/184.1; 424/85.1; 424/85.5; 424/85.7; 424/85.6

(57) ABSTRACT

The disclosure relates to a vaccine including at least one adjuvant useful in the prevention and treatment of blood cancers, for example lymphoma, leukaemia or myeloma.

The disclosure relates to a vaccine useful in the prevention and treatment of blood cancers such as lymphoma, leukaemia or myeloma.

Cancer is an abnormal disease state in which uncontrolled proliferation of one or more cell populations interferes with normal biological function. The proliferative changes are usually accompanied by other changes in cellular properties, including reversion to a less organised state. Cancer cells are typically referred to as “transformed”. Transformed cells generally display several of the following properties: spherical morphology, expression of foetal antigens, growth-factor independence, lack of contact inhibition, anchorage-independence, and growth to high density. Cancer cells form tumours and are referred to as “primary” or “secondary” tumours. A primary tumour results in cancer cell growth in an organ in which the original transformed cell develops. A secondary tumour results from the escape of a cancer cell from a primary tumour and the establishment of a secondary tumour in another organ. The process is referred to as metastasis and this process may be aggressive, for example as in the case of hepatoma or lung cancer.

Lymphomas are cancers that initiate in lymphocytes and form solid tumours in the lymph nodes. Lymphoma is a term that classifies a large number of lymphocyte originating cancers. For example B cell tumours such as chronic lymphocyte leukaemia, B cell prolymphocytic leukaemia, Waldenstrom macroglobulinemia, Burkitt's lymphoma; T cell tumours such as T cell prolymphocytic leukaemia, NK cell leukaemia, T cell large granular lymphocytic leukaemia, adult T cell leukaemia. In addition lymphoma includes the classical Hodgkin's lymphomas which themselves can be sub-divided and Non-Hodgkin lymphoma. In addition to the above, lymphomas associated with immunodeficiency are prevalent, for example those associated with HIV infection, post transplantation lymphomas and those associated with methotrexate treatment. These latter lymphomas present particular difficulties since vaccination is not a viable therapy.

The treatment of lymphoma is typically chemotherapy and radiotherapy and depending on the particular lymphoma and stage this can be effective treatment. The provision of a vaccine that protects immunodeficient subjects would be desirable. At the moment there is no validated and effective means to vaccinate against this class of cancer.

The region of an antibody that determines the binding specificity of the antibody for its antigen is referred to as the complementarity determining region (CDR) and is also referred to as the “hypervariable region” or the “idiotype”. Because the antigen binding regions of antibodies are made up of amino acid sequences derived at random they are unique to one clone or a small number of clones of B cells. These unique peptide sequences are therefore antigenic in their own right and in combination serve to make up the antibody molecule's unique idiotype. As an antigen is made up of a number of epitopes, so also an idiotype is made up of a number of “idiotopes”. Immunisation with purified immunoglobulin of a particular idiotype can generate antibody responses against that idiotype.

There are two systems that use immunoglobulins as vaccine antigens, with the aim of inducing an immune response against the immunoglobulin. In both systems it is the hypervariable region or idiotype of the antibody that is used to provoke an immune response. In both cases the idiotype of the antibody is used to generate an anti-idiotype response (anti-Id). In the first case, the idiotype of the antibody is the actual target of the immune effector response. For instance B cell lymphomas and leukemias are generally derived from a single clone of B cells and thus may express on their cell surface an immunoglobulin which is unique or almost unique to the tumour. The generation of an immune response against this immunoglobulin idiotype is the desired effect of vaccination, which may aid in clearance of the tumour cells. The anti-idiotype response generated can consist of both antibody and T cell mediated responses. Immunoglobulin idiotypes can thus be one of the best examples of a tumour specific antigen. The second system uses a so called “internal image anti-idiotype antibody” to generate a response which cross-reacts with an antigen, which may be a tumour antigen or an antigen from a pathogen or another source which for one reason or another is difficult to purify or is poorly immunogenic when administered directly.

Idiotype based vaccines, including anti-idiotype vaccines as described above, are known in the art. However, these vaccines have associated problems. Firstly, for lymphoma patients the vaccines must be individually produced as the idiotype is likely to be unique to that individual's tumour and it can take up to several months to formulate the vaccine which often involves producing hybridomas secreting the desired idiotype, purifying the immunoglobuilin and then conjugating to a protein carrier like KLH. Secondly, human immunoglobulins are inherently poorly immunogenic in humans, so despite conjugation to a carrier to augment the immune response to the idiotype, anti-Id antibody responses tend to be weak (in fact a large proportion of the response to the conjugates is directed at the highly immunogenic carrier protein. Thirdly, it has been shown in both mice and humans that both CD4+ T cells and CD8+ CTL responses against the idiotype protein may be important in mediating the therapeutic response and conjugation to a carrier such as KLH is not the most efficient means of generating CTL responses.

This disclosure relates to a vaccine and treatment regime for the prophylactic and therapeutic treatment of lymphoma.

According to an aspect of the invention there is provided a vaccine comprising:
i) an idiotype antigen isolated from a patient suffering from a lymphoma;
ii) a CD40 monoclonal antibody adjuvant, or CD40 binding fragment thereof linked to said idiotype antigen; and
iii) a second adjuvant that enhances the immune response to the linked idiotype antigen and CD40 monoclonal antibody adjuvant.

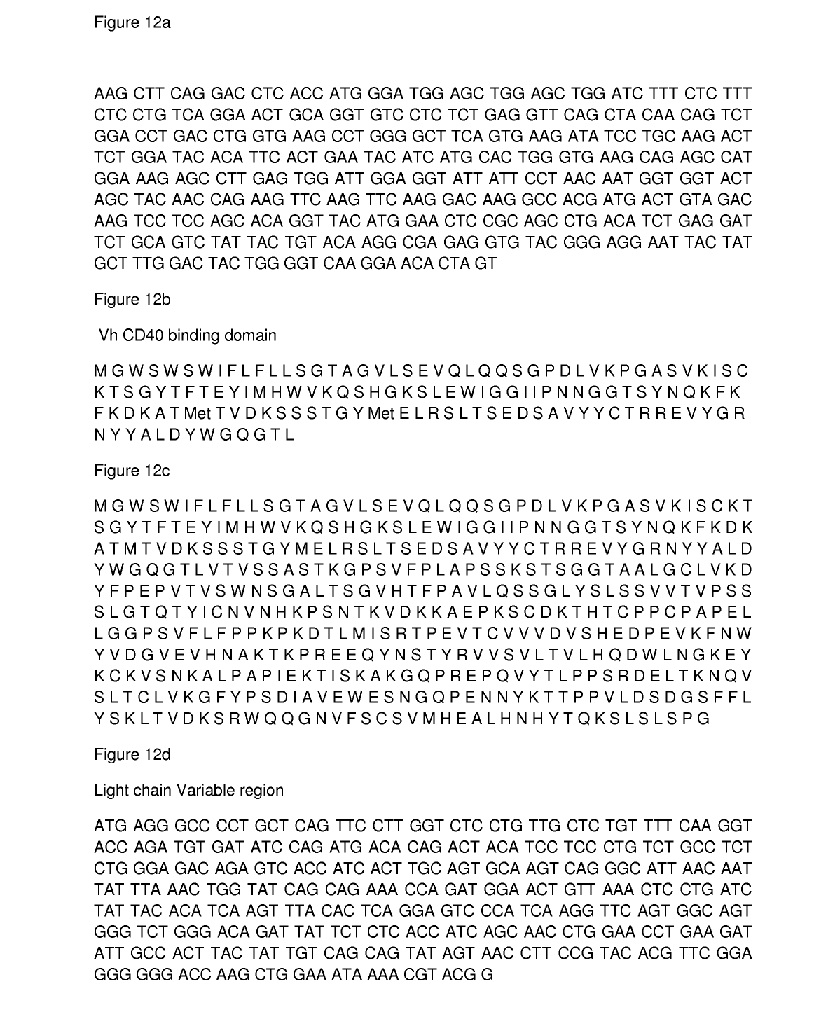

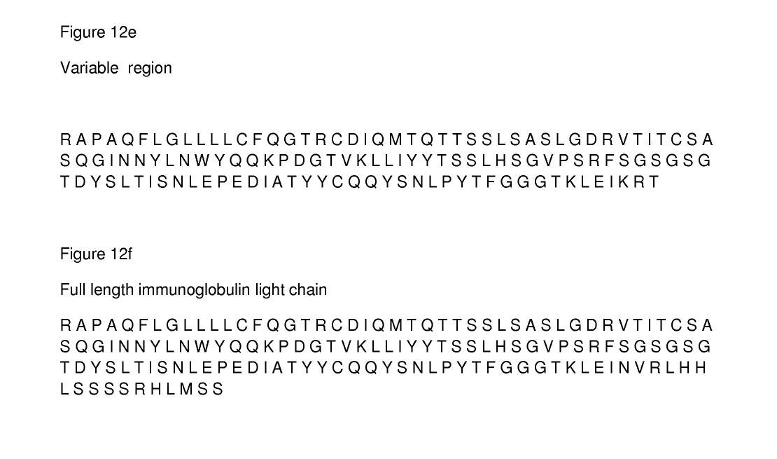

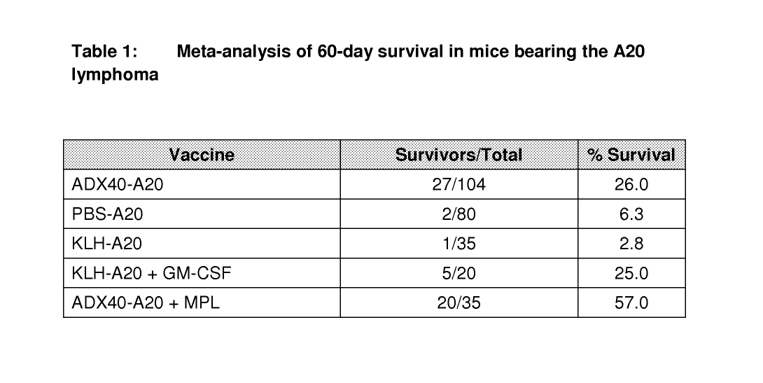

CD40 monoclonal antibodies are known in the art. For example US2009/007471 [the content of which is incorporated by reference in its entirety and specifically the amino acid sequence of the variable regions of said antibody] discloses humanized and chimeric anti-human CD40 suitable for use in the vaccine according to the invention and is represented by the sequences disclosed in FIG. 12.

In a preferred embodiment of the invention said second adjuvant is selected from the group consisting of: cytokines selected from the group consisting of GMCSF, interferon gamma, interferon alpha, interferon beta, interleukin 12, interleukin 23, interleukin 17, interleukin 2, interleukin 1, TGF, TNFα, and TNFβ.

In a further alternative embodiment of the invention said adjuvant is a TLR agonist such as CpG oligonucleotides, flagellin, monophosphoryl lipid A, polyinosinic: polycytidylic acid [poly I:C] and derivatives thereof.

In a preferred embodiment of the invention said adjuvant is poly I:C.

Poly I:C is an adjuvant that binds with toll-like receptor TLR3 which is expressed by B cells and dendritic cells.

In a preferred embodiment of the invention said adjuvant is a bacterial cell wall derivative such as muramyl dipeptide (MDP) and/or trehelose dycorynemycolate (TDM) and/or monophosphophoryl lipid A [MPL].

In a preferred embodiment of the invention said adjuvant is MPL.

An adjuvant is a substance or procedure which augments specific immune responses to antigens by modulating the activity of immune cells. Examples of adjuvants include, by example only, agonistic antibodies to co-stimulatory molecules, Freund's adjuvant, muramyl dipeptides, liposomes, alum, QS21. An adjuvant is therefore an immunomodulator. A carrier is an immunogenic molecule which, when bound to a second molecule augments immune responses to the latter. The term carrier is construed in the following manner. A carrier is an immunogenic molecule which, when bound to a second molecule augments immune responses to the latter. Some antigens are not intrinsically immunogenic yet may be capable of generating antibody responses when associated with a foreign protein molecule such as keyhole-limpet haemocyanin or tetanus toxoid. Such antigens contain B-cell epitopes but no T cell epitopes. The protein moiety of such a conjugate (the “carrier” protein) provides T-cell epitopes which stimulate helper T-cells that in turn stimulate antigen-specific B-cells to differentiate into plasma cells and produce antibody against the antigen. Helper T-cells can also stimulate other immune cells such as cytotoxic T-cells, and a carrier can fulfill an analogous role in generating cell-mediated immunity as well as antibodies. Certain antigens which lack T-cell epitopes, such as polymers with a repeating B-cell epitope (e.g. bacterial polysaccharides), are intrinsically immunogenic to a limited extent. These are known as T-independent antigens. Such antigens benefit from association with a carrier such as tetanus toxoid, under which circumstance they elicit much stronger antibody responses.

In a preferred embodiment of the invention said idiotype antigen comprises or consists of a Fab or F(ab)2′ Fd fragment of said idiotype immunoglobulin.

The idiotype antigen can be within the variable regions of the heavy and light immunoglobulin chains i.e. (Fab, F(ab)2′, Fd fragment or indeed chimeric between the heavy and light variable regions and another antibodies constant regions.

According to an aspect of the invention there is provided a vaccine comprising an
i) an idiotype antigen isolated from a patient suffering from a lymphoma;
ii) a CD40 monoclonal antibody adjuvant or CD40 binding fragment thereof linked to said idiotype antigen; and
iii) a second adjuvant that enhances the immune response to the linked idiotype antigen and CD40 monoclonal antibody adjuvant for use in the treatment of lymphoma.

In a preferred embodiment of the invention said lymphoma is B cell lymphoma.

In a preferred embodiment of the invention said B cell lymphoma is selected from the group consisting of: chronic lymphocyte leukaemia, B cell prolymphocytic leukaemia, Burkitt's lymphoma, follicular lymphoma, myeloma .

B cell acute lymphoblastic leukaemia, Chronic lymphocytic leukemia/Small lymphocytic lymphoma, B-cell prolymphocytic leukemia, Lymphoplasmacytic lymphoma (such as Waldenström macroglobulinemia), Splenic marginal zone lymphoma, Plasma cell neoplasms, Plasma cell myeloma, Plasmacytoma, Extranodal marginal zone B cell lymphoma, also called MALT lymphoma, Nodal marginal zone B cell lymphoma (NMZL, Follicular lymphoma, Mantle cell lymphoma, Diffuse large B cell lymphoma, Mediastinal (thymic) large B cell lymphoma, Intravascular large B cell lymphoma, Primary effusion lymphoma, Burkitt lymphoma/leukemia.

In a further preferred embodiment of the invention said lymphoma is Hodgkin's lymphoma.

In an alternative preferred embodiment of the invention said lymphoma is non Hodgkin's lymphoma.

In a further preferred embodiment of the invention said lymphoma is an immunodeficiency associated lymphoma.

In a preferred embodiment of the invention said immunodeficiency associated lymphoma is HIV associated.

In a preferred embodiment of the invention said immunodeficiency associated lymphoma is transplantation associated.

In a further preferred embodiment of the invention said immunodeficiency associated lymphoma is the result of methotrexate treatment.

According to a further aspect of the invention there is provided a vaccine comprising:
i) an idiotype antigen isolated from a patient suffering from myeloma;
ii) a CD40 monoclonal antibody adjuvant, or CD40 binding fragment thereof linked to said idiotype antigen; and
iii) a second adjuvant that enhances the immune response to the linked idiotype antigen and CD40 monoclonal antibody or CD40 binding fragment thereof adjuvant for use in the treatment of myeloma.

When administered the vaccines or pharmaceutical composition comprising the vaccine of the present invention are administered in pharmaceutically acceptable preparations. Such preparations may routinely contain pharmaceutically acceptable concentrations of salt, buffering agents, preservatives, compatible carriers, supplementary immune potentiating agents and optionally other therapeutic agents, such as chemotherapeutic agents which can be administered separately from the vaccines of the invention or in a combined preparation if a combination is compatible. The vaccines of the invention can be administered by any conventional route, including injection or by gradual infusion over time. The administration may, for example, be oral, intravenous, intraperitoneal, intramuscular, intracavity, subcutaneous, or transdermal.

The compositions of the invention are administered in effective amounts. An “effective amount” is that amount of a composition that alone, or together with further doses, produces the desired immunological response. In the case of treating lymphoma the desired response is inhibiting the progression of the disease. This may involve only slowing the progression of the disease temporarily, although more preferably, it involves halting the progression of the disease permanently. This can be monitored by routine methods.

The vaccines used in the foregoing methods preferably are sterile and contain an effective amount for producing the desired response in a unit of weight or volume suitable for administration to a patient. The response can, for example, be measured by determining regression of a tumour, decrease of disease symptoms, modulation of apoptosis, etc.

Other protocols for the administration of vaccines will be known to one of ordinary skill in the art, in which the dose amount, schedule of injections, sites of injections, mode of administration (e.g. intra-tumoral) and the like vary from the foregoing. Administration of compositions to mammals other than humans, e.g. for testing purposes or veterinary therapeutic purposes, is carried out under substantially the same conditions as described above. A subject, as used herein, is a mammal, preferably a human, and including a non-human primate, cow, horse, pig, sheep, goat, dog, cat or rodent.

According to a further aspect of the invention there is provided a method to manufacture a vaccine suitable for prophylaxis or treatment of lymphoma comprising:
i) isolating from a subject that has or is susceptible to a lymphoma an idiotype antigen;
ii) linking said idiotype antigen with a CD40 monoclonal antibody adjuvant or CD40 binding fragment thereof to form a complex and optionally isolating the linked complex; and
iii) forming a preparation of the linked antigen/adjuvant complex with at least one additional adjuvant.

According to a further aspect of the invention there is provided a method to manufacture a vaccine suitable for prophylaxis or treatment of lymphoma comprising:
i) providing an isolated biological sample comprising a lymphoma cell;
ii) providing an isolated hybridoma cell that produces a CD40 monoclonal antibody or CD40 binding fragment thereof;
iii) forming a preparation suitable for promoting the fusion of the lymphoma cell with said hybridoma cell line to form a hybrid cell;
iv) screening said hybrid cells for monoclonal antibodies wherein said antibodies comprise at least two immunoglobulins or antigen binding part thereof wherein one immunoglobulin or part is specific for CD40 and a second immunoglobulin or part is a lymphoma idiotype; and
v) forming a preparation of the linked antigen/adjuvant complex with at least one additional adjuvant.

In a preferred method of the invention said second adjuvant is selected from the group consisting of: cytokines selected from the group consisting of GMCSF, interferon gamma, interferon alpha, interferon beta, interleukin 12, interleukin 23, interleukin 17, interleukin 2, interleukin 1, TGF, TNFα, and TNFβ.

In a further alternative method of the invention said adjuvant is a TLR agonist such as CpG oligonucleotides, flagellin, monophosphoryl lipid A, poly I:C and derivatives thereof.

In a preferred method of the invention said adjuvant is poly I:C.

In a preferred method of the invention said adjuvant is a bacterial cell wall derivative such as muramyl dipeptide (MDP) and/or trehelose dycorynemycolate (TDM) and/or monophosphophoryl lipid A [MPL].

In a preferred method of the invention said adjuvant is MPL.

According to an aspect of the invention there is provided a vaccine comprising:
i) an idiotype antigen isolated from a patient suffering from a lymphoma; and
ii) an adjuvant that enhances the immune response to the idiotype antigen for use in the treatment of lymphoma.

In a preferred embodiment of the invention said adjuvant is selected from the group consisting of: cytokines selected from the group consisting of GMCSF, interferon gamma, interferon alpha, interferon beta, interleukin 12, interleukin 23, interleukin 17, interleukin 2, interleukin 1, TGF, TNFα, and TNFβ.

In a further alternative preferred embodiment of the invention said adjuvant is a TLR agonist such as CpG oligonucleotides, flagellin, monophosphoryl lipid A, poly I:C and derivatives thereof.

In a preferred embodiment of the invention said adjuvant is poly I:C.

In a preferred embodiment of the invention said adjuvant is a bacterial cell wall derivative such as muramyl dipeptide (MDP) and/or trehelose dycorynemycolate (TDM) and/or monophosphophoryl lipid A [MPL].

In a preferred embodiment of the invention said adjuvant is MPL.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

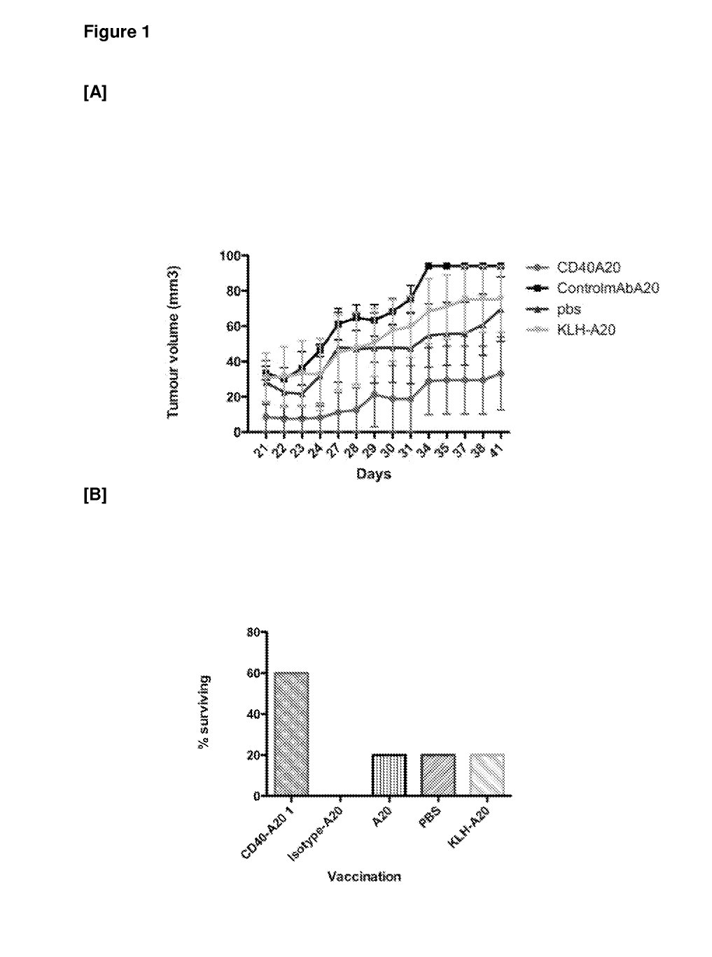

FIG. 1 Tumour growth [A] and Day 60 survival [B] of A20 lymphoma in mice (n=5 per group) following 2 doses of vaccine given 2 months apart;

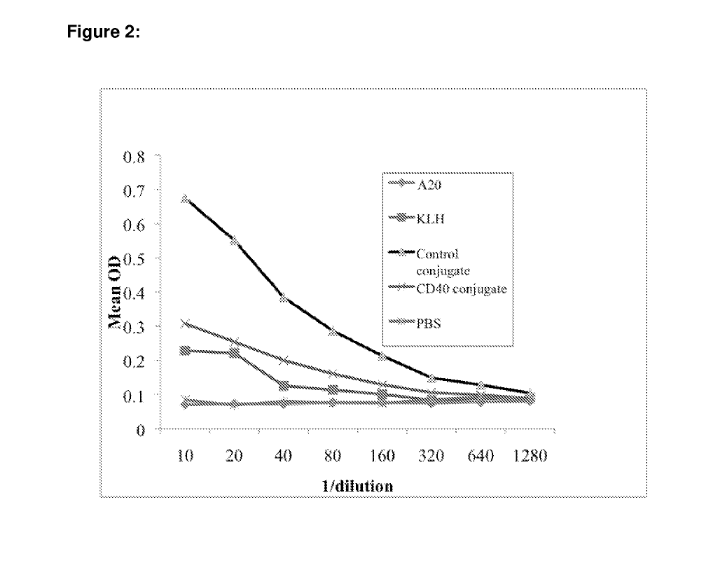

FIG. 2 IgG anti-A20 antibody response 14 days after the second vaccination and prior to tumour challenge;

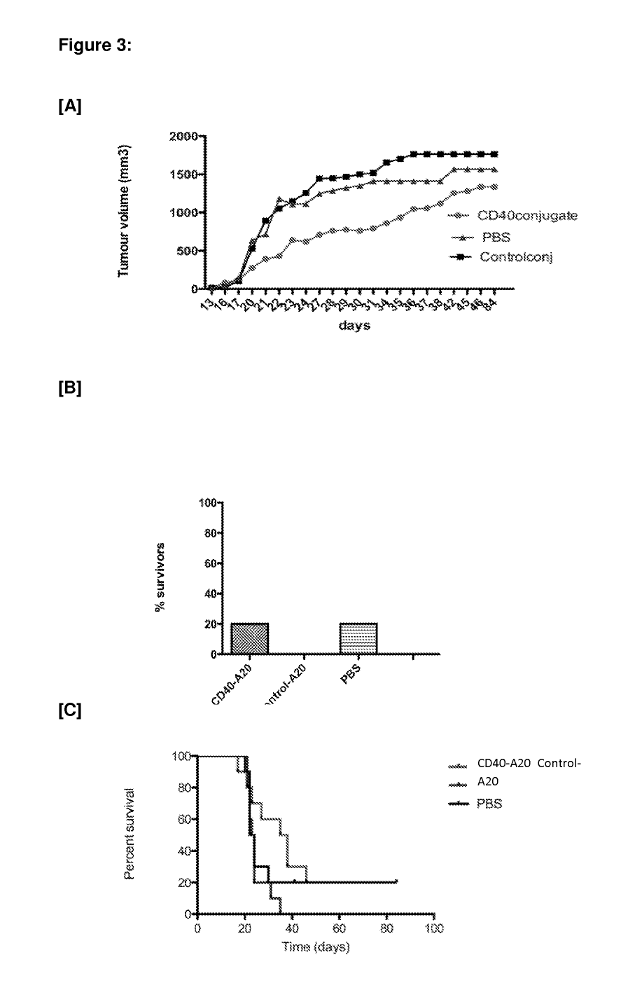

FIG. 3 IgG anti-A20 antibody response 14 days after the second vaccination and prior to tumour challenge;

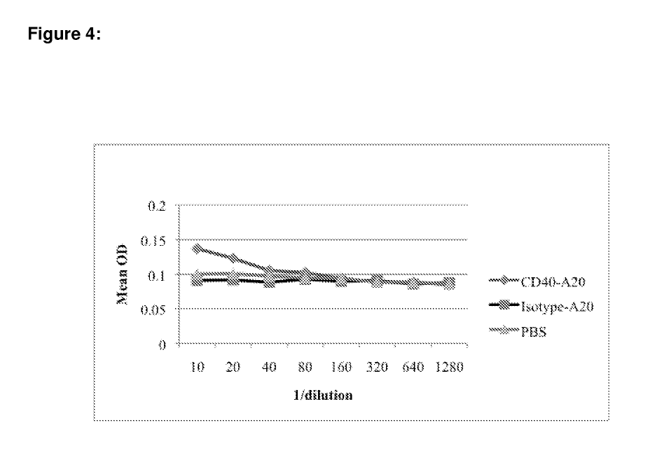

FIG. 4 IgG anti-A20 antibody response 14 days after a single vaccination and prior to tumour challenge;

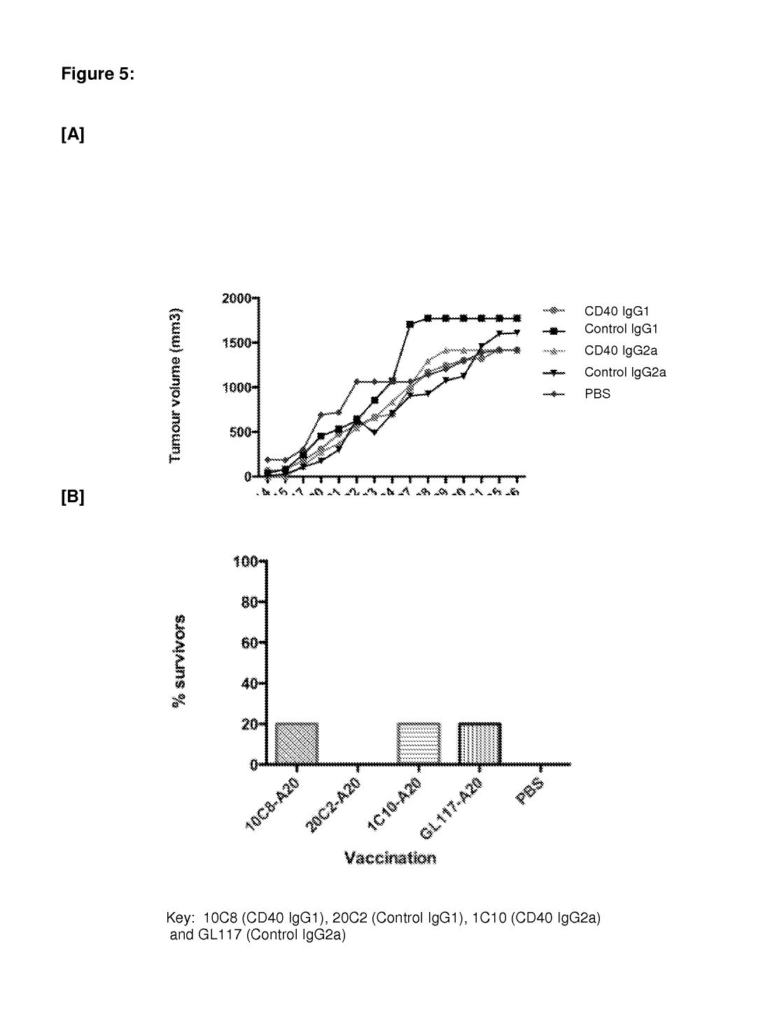

FIG. 5 Tumour growth [A] and Day 60 survival [B] and survival of A20 lymphoma in mice (n=5 per group) following a single dose of IgG1 and IgG2a isotypes of ADX40-A20 conjugate vaccines;

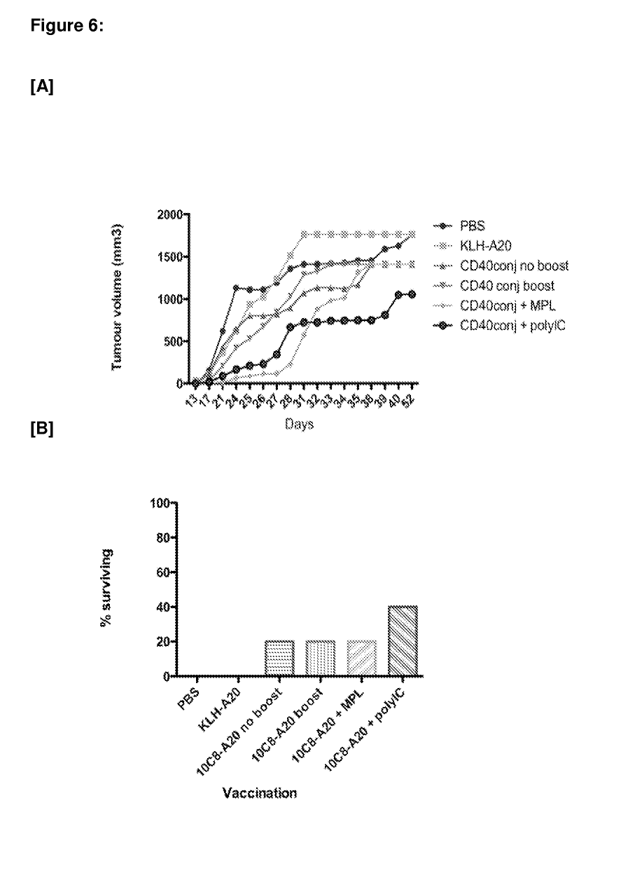

FIG. 6 Tumour growth [A] and Day 60 survival [B] of A20 lymphoma in mice (n=5 per group) following 2 doses of vaccine given 2 weeks apart;

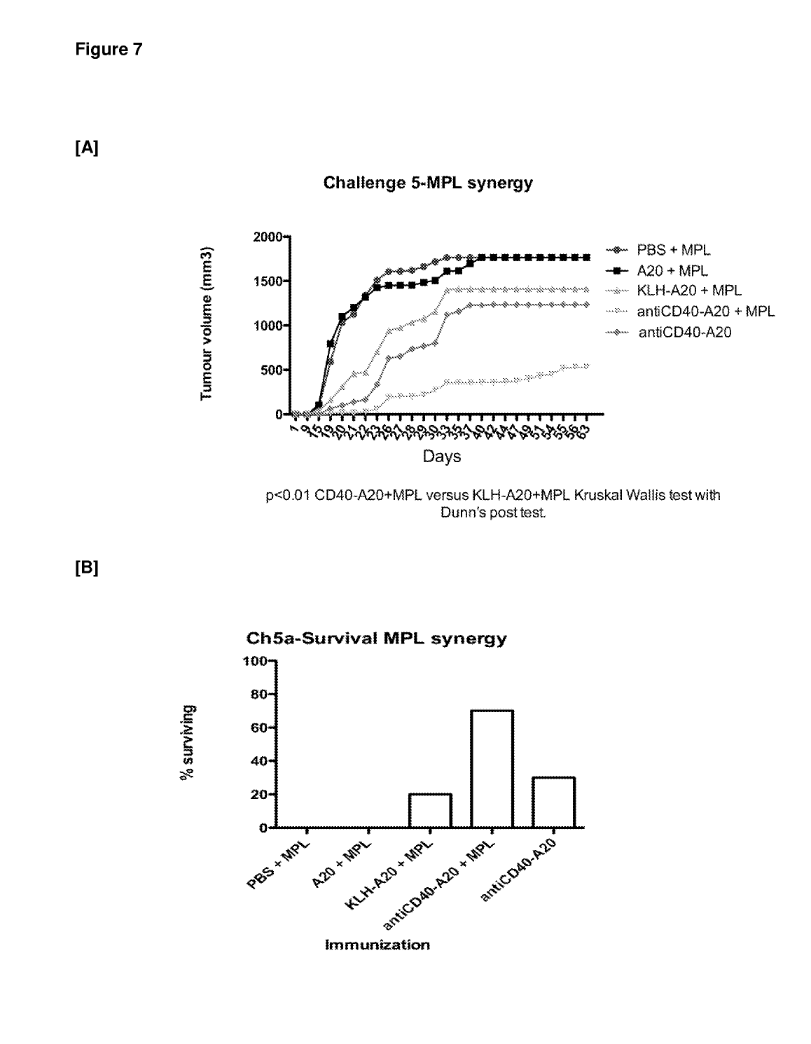

FIG. 7 Tumour growth [A] and Day 60 survival [B] of A20 lymphoma in mice (n=5 per group) following 2 doses of vaccine given 2 weeks apart with or without MPL [Challenge 5a];

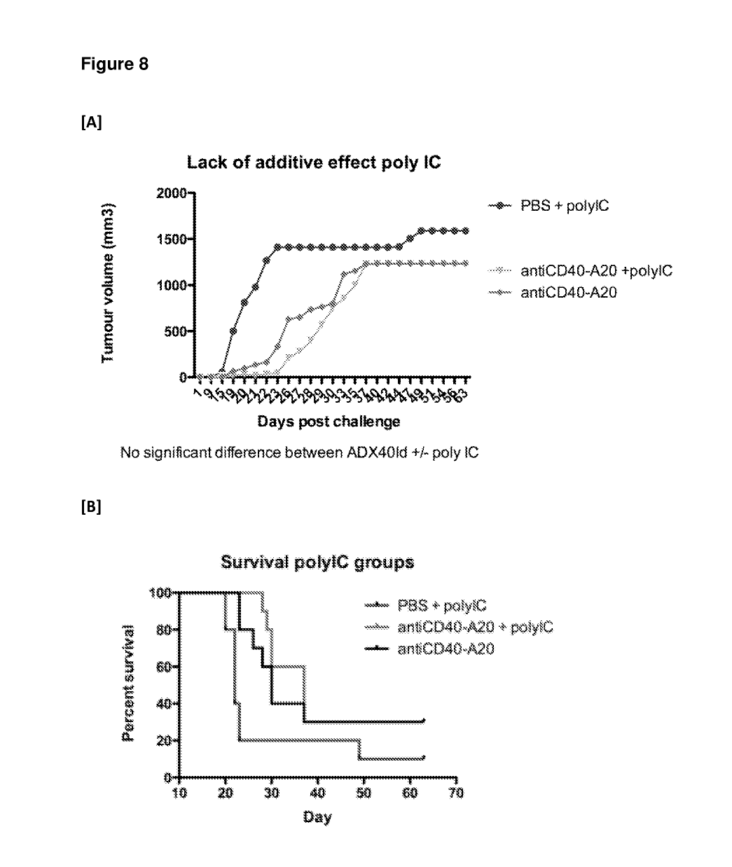

FIG. 8 Tumour growth [A] and survival curves [B] of A20 lymphoma in mice (n=10 per group) following 2 doses of vaccine given 2 weeks apart with or without poly-I:C [Challenge 5b];

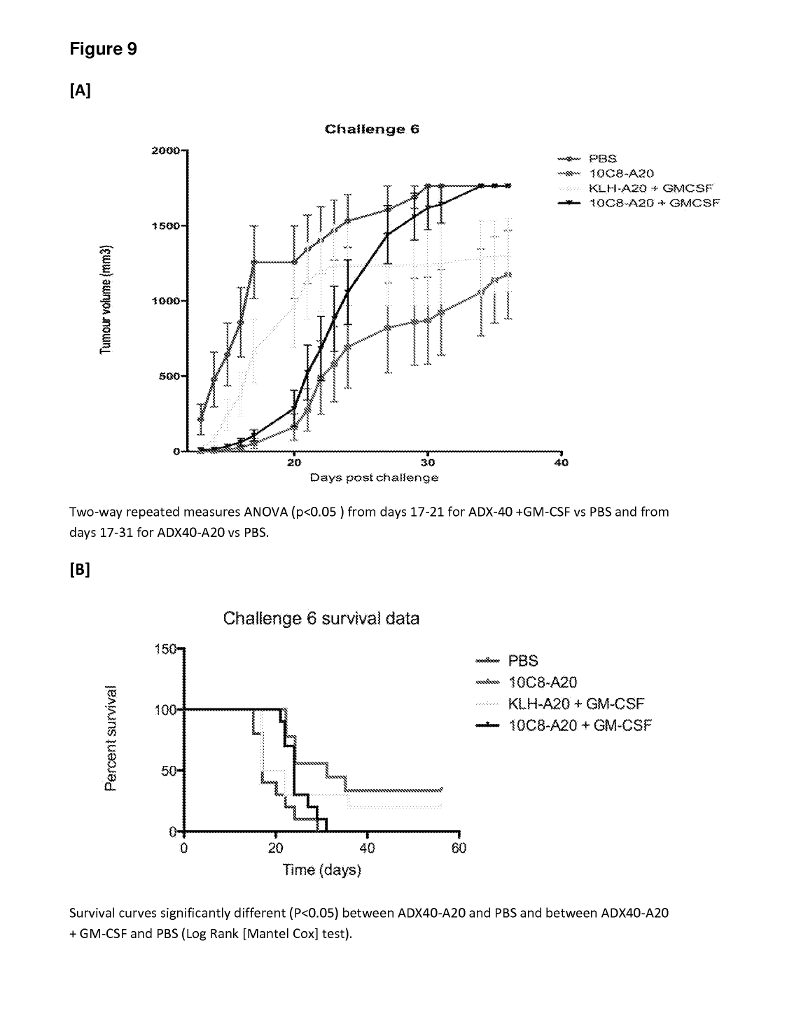

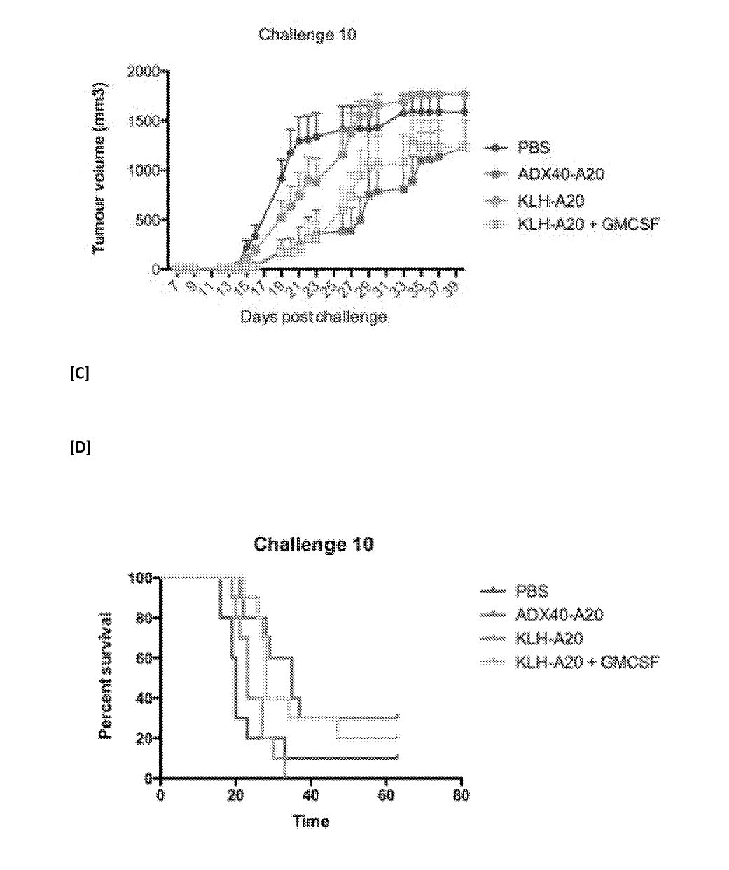

FIG. 9 Tumour growth [A,C] and survival [B, D] curves of A20 lymphoma in mice (n=10 per group) following 2 doses of vaccine given together with GM-CSF [Challenges 6 and 10

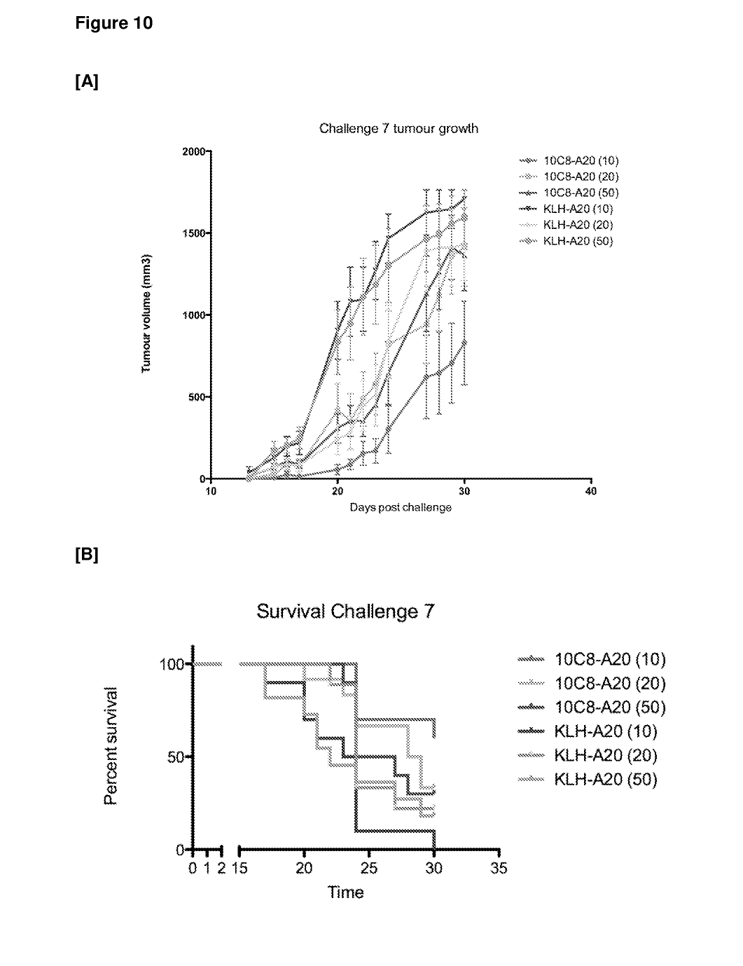

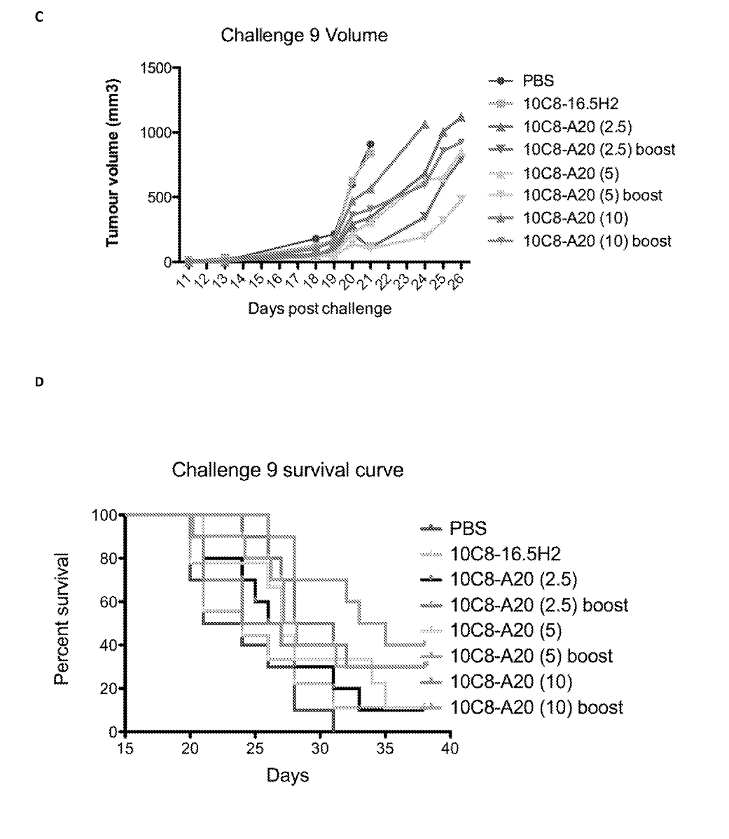

FIG. 10 Tumour growth [A,C] and survival curves [B,D] of A20 lymphoma in mice (n=10 per group) following increasing dose-levels of ADX40-A20 or KLH-A20 conjugate [Challenges 7 and 9];

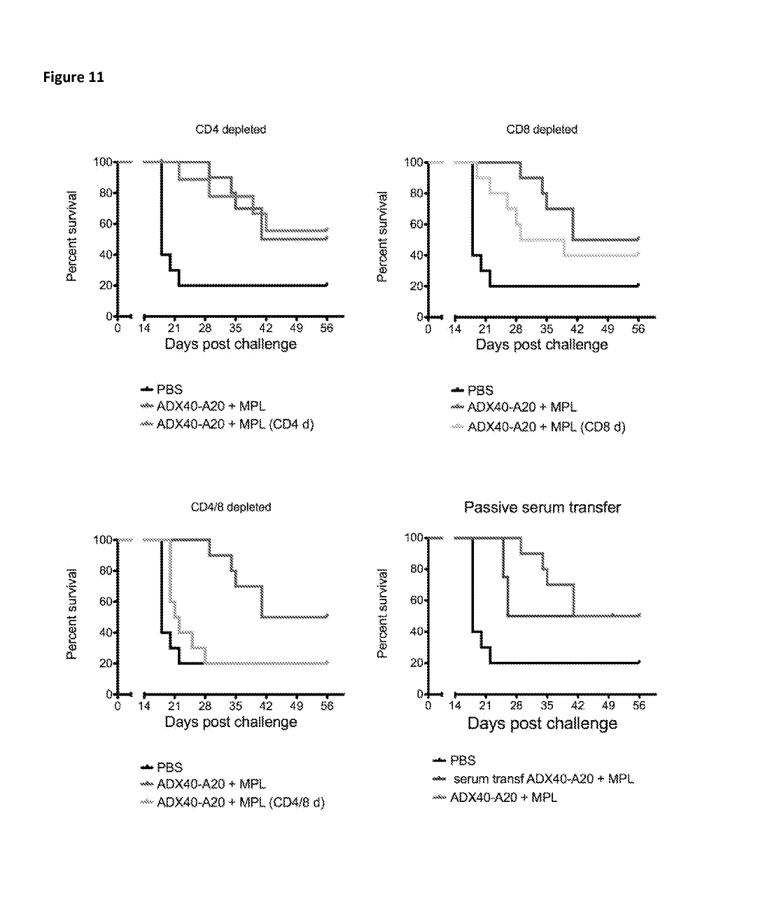

FIG. 11 illustrates a potential mode of action of the ADX40 lymphoma vaccine; and

FIG. 12a is the nucleotide sequence of variable heavy chain of a CD40 monoclonal antibody that binds human CD40; FIG. 12b is the amino acid sequence of the CD40 monoclonal antibody that binds human CD40; FIG. 12c is a chimeric antibody comprising the CD40 variable region fused to a human IgG1 constant region heavy chain; FIG. 12d is the nucleotide sequence of the variable light chain CD40 monoclonal antibody that binds human CD40; FIG. 12e is the amino acid sequence of the variable light chain CD40 monolconal antibody that binds human CD40; and FIG. 12f is the full length variable light chain referred to in FIG. 12e

### MATERIALS AND METHODS

Production of idiotype protein for conjugation

Tumour idiotype protein for incorporation into lymphoma or leukaemia vaccines can be obtained by one of two broad methods. In both cases tumour cells are first obtained by biopsy.

The first method is hybridoma rescue, wherein the tumour cells are fused with a human/mouse heterohybridoma or a similar immortalised cell line such as a myeloma line, to produce further hybridomas which are then selected for based on secretion of the tumour idiotype protein. The selected hybridomas are then grown in tissue culture, usually in bioreactors, to obtain supernatant from which the idiotype protein is purified ^{1 2 3}

The second method is recombinant Id production. In this cDNA sequences of both heavy and light chain variable regions of the Id-containing, tumour-specific immunoglobulin are cloned by standard molecular biological methods such as PCR. Both cloned sequences are then inserted into a plasmid that contains the sequence of a shared immunoglobulin constant region, and the complete plasmid is finally transduced into different living hosts such as bacteria, mammalian cells, insect cells, yeast cells or tobacco plants. The Id protein is later purified from culture supernatant or the transfected cells ⁴.

Preparation of Conjugate

A20 idiotype protein (A20) was purified by protein G affinity chromatography from the bioreactor supernatant of a rescue hybridoma between A20 lymphoma cells and P3X63 myeloma cells prepared by PEG fusion. A20 idiotype protein was first reacted with sulpho-succinimidyl 4-[N-maleimidedomethyl]-cyclohexane-1-carboxylate (sulpho-SMCC) to produce maleimide-activated A20. Murine IgG1 or IgG2a anti-CD40 monoclonal antibodies (both ADX40; IgG1 Variant and IgG2a Variant) were meanwhile treated with N-succinimidyl S-acetylthioacetate (SATA), to introduce a protected sulphydryl group. Subsequent deacetylation of the antibodies with hydroxylamine generated a free sulphydryl group, which reacted with the maleimide group on A20 to form stable conjugates. Isotype-matched control antibodies were conjugated to A20 in a similar manner.

Cross-linking was confirmed by SDS-polyacrylamide gel electrophoresis and Western blotting.

Immunisation and Challenge of Mice

6-8 week old female BALB/c mice were given one or two intraperitoneal injections of immunogen followed, at least 2 weeks after the last immunization, with a subcutaneous challenge using 10⁵ A20 lymphoma cells. Tumour growth was monitored to 15mm diameter, at which point the mice were culled. Survival curves show non-surviving mice as those in which tumours had reached the 15mm threshold. Survival at 60 days represents the percentage of mice in which no visible tumours had appeared.

Control vaccinations included an isotype-matched control conjugate, A20 conjugated to keyhole limpet haemocyanin (KLH), A20 antigen alone or phosphate buffered saline (PBS).

Humoral immune response

IgG against Fab fragments of A20 were measured by enzyme-linked immunosorbent assay (ELISA) using a rat anti-mouse IgG Fc specific detection antibody (Jackson Immuno Research Laboratories).

### Example 1

Effect of Two Vaccinations (Challenge 1)

Tumour volume and survival data from mice (5 per group) vaccinated two months apart with the ADX40 IgG1 variant and challenged 20 days after the second vaccination are shown in FIGS. 1A and 1B respectively.

Tumour outgrowth in the ADX40-A20 conjugate group was significantly lower than in animals vaccinated with the isotype control, KLH-A20 conjugate or A20 alone (p<0.01 by

Kruskal-Wallis test using Dunn's post-test correction) and was also significantly lower than in the PBS group (p<0.05).

Day 60 survival of the ADX40-A20 conjugate immunised group was significantly better than with the isotype control immunised group (p=0.048, Fisher's exact test) but although survival was greater than in animals given KLH-A20 conjugate, A20 antigen alone or PBS, this did not reach statistical significance.

FIG. 2 shows the anti-A20 antibody response induced 14 days after the second vaccination. The isotype control conjugate was highly immunogenic in terms of antibody induction, but this did not translate into decreased tumour growth or improved survival. Antibody responses were also observed to the ADX40-A20 and KLH-A20 conjugates, but at a lower level than for the isotype control, suggesting prevention of tumour outgrowth is only partly reflected in the antibody response.

### Example 2

Effect of a Single Vaccination (Challenge 2)

Tumour volume and survival data from mice (10 per group) vaccinated on a single occasion with the ADX40 IgG1 Variant conjugate and challenged 14 days later are shown in FIGS. 3A-C.

Tumour outgrowth in the ADX40-A20 conjugate group was significantly lower and slower than in both control groups (PBS p<0.05; control conjugate p<0.001; Kruskal-Wallis test with Dunn's post correction). However, survival at Day 60 was not significantly better in the ADX40-A20 conjugate treated group than in the PBS control group, although survival was better in the conjugate group at earlier time-points.

Single immunisation with conjugates resulted in a slightly stronger antibody response in the ADX40-A20 treated group than in control animals (FIG. 4).

### Example 3

Comparison of IgG1 and IaG2a ADX40 Isotypes (Challenge 3)

Tumour outgrowth and Day 60 survival data comparing the ADX40 IgG1 Variant and the ADX40 IgG2a Variant each given as a single vaccination 14 days prior to challenge are shown in FIGS. 5A and 5B respectively.

There was no statistically significant difference in tumour outgrowth or Day 60 survival between either ADX40 Variant or controls (Kruskal-Wallis and Chi squared tests), although there was a trend towards early slower tumour growth and increased Day 60 survival with both ADX40 conjugate Variants when compared with the PBS control group.

### Example 4

Evaluation of Combined Adjuvants (Challenge 4)

The effect of combining the adjuvants MPL or poly-I:C to the ADX40 IgG1 Variant-A20 conjugate vaccine was explored in a challenge study in which mice (5 per group) were vaccinated twice, 2 weeks apart, followed by challenge two weeks later. Tumour outgrowth and survival data are shown in FIG. 6A and 6B respectively.

Neither one nor two doses of conjugate led to significant differences in tumour growth versus either PBS or KLH conjugate groups. ADX40 plus MPL significantly slowed tumour growth compared with KLH conjugate (p<0.01) and PBS (p<0.05). ADX40 plus poly I:C significantly slowed tumour growth in comparison with both PBS and KLH groups (p<0.01). All comparisons by Kruskal-Wallis test with Dunn's post-test.

ADX40 alone, given once or twice, did not significantly improve survival at Day 60 in comparison with KLH or PBS groups.

### Example 5

The experiments with MPL (Challenge 5a) and poly-I:C (Challenge 5b) were repeated with larger groups of mice (10 per group) and the data are shown in FIGS. 7 and 8 respectively.

ADX40-A20+MPL immunised mice had significantly slower tumour growth than KLH-A20+MPL immunised mice (P<0.01, Kruskal-Wallis test). There was a clear trend towards an additive effect of MPL and ADX40, although this was not statistically significant at this time.

Unlike MPL however, in the second challenge poly-I:C did not add significantly to the antitumour efficacy of ADX40-A20, although there was a trend towards an adjuvant effect of the adjuvant when combined with the KLH-A20 conjugate.

### Example 6

Comparison with “clinical” regimen (Challenges 6 and 10a)

To compare the effect of A20 conjugated to ADX40 with a regimen analogous to that used with clinical idiotype lymphoma vaccines (i.e. idiotype antigen conjugated to KLH and adjuvanted with GM-CSF), groups of 10 mice were given two injections (14 days apart) of ADX40-A20, ADX40 +GM-CSF and KLH-A20 +GM-CSF. The GM-CSF was given subcutaneously at a dose of 55ng per mouse for 4 consecutive days starting with the day of vaccination (i.e. analogous to the clinical regimen). The tumour volume and survival data are shown in FIGS. 9A and 9B respectively.

Tumour growth was significantly delayed in the ADX40 conjugate +GM-CSF (to day 21) and ADX40 conjugate groups (to day 31), indicating that ADX40 conjugate is superior to the “clinical” vaccine analogue.

ADX40 conjugate and ADX40 conjugate +GM-CSF had a significant survival advantage over the control PBS group (p=0.001 and p <0.03 respectively. Median survival of the control PBS group was 17 days. This increased to 19.5 days for KLH conjugate +GM-CSF, and to 24 and 31 days for ADX40 conjugate +GM-CSF and ADX40 conjugate, respectively. Data from Challenge 10a (FIGS. 9C and 9D) supported these observations.

### Example 7

In order to optimise conjugate doses for confirmatory experiments, groups of 10 mice were given 10, 20 or 50 μg of ADX40-A20 or KLH-A20 conjugate. The tumour volume and survival data are shown in FIG. 10.

The lowest dose (10 μg) of ADX40-A20 conjugate and the intermediate dose (20 μg) of KLH-A20 conjugate were found to be the most effective. A further experiment with an even lower dose range of ADX40 was then performed, with one or two doses of 10, 5 and 2.5 μg conjugate. 5 μg ADX40 conjugate appeared to be the most effective dose, whether given just once, or twice (FIG. 10). Two doses of conjugate, irrespective of dose, not unexpectedly, generated a better outcome than one dose. Finally, a control conjugate of ADX40 with a different mouse idiotype protein induced no protection, showing specificity of the protection induced by the ADX40 vaccine.

### Example 8

Two therapeutic vaccination studies were carried out in which ADX40-A20 conjugate or KLH-A20 +GM-CSF was given 3 days (first experiment) or 3 and 11 days (second experiment) after subcutaneous implantation of tumour. None of vaccine regimens had a significant effect on tumour outgrowth or survival compared with controls (data not shown). These data are not surprising, since in the clinical setting idiotype vaccination is generally used in patients with minimum residual disease who are in remission following chemotherapy.

### Example 9

A meta-analysis of survival data from the various challenge experiments is shown in Table 1. Mice given ADX40-A20 had significantly improved survival compared with those given KLH-A20 (26% versus 2.8% respectively; p=0.007). Furthermore, 60-day survival of mice given ADX40-20 was similar to that in mice given the current clinical regimen of KLH-A20 plus GM-CSF (25%), but with 2 rather than 8 injections. Finally, there is evidence to suggest synergistic effects can be seen when additional adjuvants are combined with ADX40 conjugates, since the survival of animals given ADX40-A20 plus MPL was significantly greater than with other groups.

### Example 10

Example 10 illustrates a potential mode of action. Mice were immunised with ADX40-A20+MPL and just prior to challenge depleted of either CD4 T cells, CD8 T cells, or both. Depletion of CD4 cells alone had no obvious effect on tumour growth or survival rates. Depletion of CD8 T cells decreased protection, indicating a likely role for CD8 T cells in mediating ADX40+MPL protection. Depletion of CD4 and CD8 cells appeared to decrease protection further; FIG. 11.

### REFERENCES

1. Carroll W L, Thielemans K, Dilley J, Levy R. Mouse x human heterohybridomas as fusion partners with human B cell tumors. J Immunol Methods. 1986;89:61-72.
2. Rodriguez-Calvillo M, Inoges S, Lopez-Diaz de Cerio A, Zabalegui N, Villanueva H, Bendandi M. Variations in “rescuability” of immunoglobulin molecules from different forms of human lymphoma: implications for anti-idiotype vaccine development. Crit Rev Oncol Hematol. 2004;52:1-7.
3. Kwak L W, Campbell M J, Czerwinski D K, Hart S, Miller R A, Levy R. Induction of immune responses in patients with B-cell lymphoma against the surface-immunoglobulin idiotype expressed by their tumors. N Engl J Med. 1992;327:1209-1215.
4. Park H J, Neelapu S S. Developing idiotype vaccines for lymphoma: from preclinical studies to phase III clinical trials. Br J Haematol. 2008;142:179-191.

1. A vaccine composition comprising:
i) an idiotype antigen isolated from a patient suffering from a lymphoma or leukaemia
ii) a CD40 monoclonal antibody or CD40 antibody binding fragment linked to said idiotype antigen; and
iii) an adjuvant that enhances the immune response to the linked idiotype antigen and CD40 monoclonal antibody or CD40 binding antibody fragment.

2. A vaccine according to claim 1 wherein said adjuvant is a cytokine selected from the group consisting of GMCSF, interferon gamma, interferon alpha, interferon beta, interleukin 12, interleukin 23, interleukin 17, interleukin 2, interleukin 1, TGF, TNFα, and TNFβ.

3. A vaccine according to claim 1 wherein said adjuvant is a TLR agonist selected from the group consisting of CpG oligonucleotides, flagellin, monophosphoryl lipid A, and poly I:C.

4. A vaccine according to claim 3 wherein said adjuvant is poly I:C.

5. A vaccine according to claim 1 wherein said adjuvant is a bacterial cell wall derivative selected from the group consisting of muramyl dipeptide (MDP), trehelose dycorynemycolate (TDM), and monophosphophoryl lipid A [MPL].

6. A vaccine according to claim 5 wherein said adjuvant is MPL.

7. A vaccine according to claim 1 wherein said idiotype antigen comprises or consists of a Fab or F(ab)2′ fragment of said idiotype antigen.

8-15. (canceled)

16. A method to manufacture a vaccine suitable for prophylaxis or treatment of lymphoma comprising:
i) isolating an idiotype antigen from a subject that has or is susceptible to a lymphoma or leukaemia;
ii) linking said idiotype antigen with a CD40 monoclonal antibody or CD40 binding fragment thereof to form a complex and optionally isolating the linked complex; and
iii) forming a preparation of the linked antigen/adjuvant complex with at least one additional adjuvant.

17. A method to manufacture a vaccine suitable for prophylaxis or treatment of lymphoma comprising:
i) providing an isolated biological sample comprising a lymphoma cell;
ii) providing an isolated hybridoma cell that produces a CD40 monoclonal antibody;
iii) forming a preparation suitable for promoting the fusion of the lymphoma cell with said hybridoma cell line to form a hybrid cell;
iv) screening said hybrid cells for monoclonal antibodies wherein said antibodies comprise at least two immunoglobulins wherein one immunoglobulin is specific for CD40 and a second immunoglobulin is a lymphoma or leukaemia idiotype; and
v) forming a preparation of the linked antigen/adjuvant complex with at least one additional adjuvant.

18. A method according to claim 16 wherein said adjuvant is a cytokine selected from the group consisting of GMCSF, interferon gamma, interferon alpha, interferon beta, interleukin 12, interleukin 23, interleukin 17, interleukin 2, interleukin 1, TGF, TNFα, and TNFβ.

19. A method according to claim 16 wherein said adjuvant is a TLR agonist selected from the group consisting of CpG oligonucleotides, flagellin, monophosphoryl lipid A, and poly I:C.

20. A method according to claim 19 wherein said adjuvant is poly I:C.

21. A method according to claim 16 wherein said adjuvant is a bacterial cell wall derivative selected from the group consisting of muramyl dipeptide (MDP), trehelose dycorynemycolate (TDM), and monophosphophoryl lipid A [MPL].

22. A composition, comprising:
i) an idiotype antigen isolated from a patient suffering from a lymphoma; and
ii) an adjuvant that enhances the immune response to the idiotype antigen for use in the treatment of lymphoma.

23. A composition according to claim 22 wherein said adjuvant is selected from the group consisting of: cytokines selected from the group consisting of GMCSF, interferon gamma, interferon alpha, interferon beta, interleukin 12, interleukin 23, interleukin 17, interleukin 2, interleukin 1, TGF, TNFα, and TNFβ.

24. A composition according to claim 22 wherein said adjuvant is a TLR agonist selected from the group consisting of CpG oligonucleotides, flagellin, monophosphoryl lipid A, poly I:C and derivatives thereof.

25. A composition according to claim 22 wherein said adjuvant is poly I:C.

26. A composition according to claim 22 wherein said adjuvant is a bacterial cell wall derivative selected from the group consisting of muramyl dipeptide (MDP), trehelose dycorynemycolate (TDM), and monophosphophoryl lipid A [MPL].

27. A composition according to claim 26 wherein said adjuvant is MPL.

28. A method according to claim 17,-wherein said adjuvant is a cytokine selected from the group consisting of GMCSF, interferon gamma, interferon alpha, interferon beta, interleukin 12, interleukin 23, interleukin 17, interleukin 2, interleukin 1, TGF, TNFα, and TNFβ.

29. A method according to claim 17-wherein said adjuvant is a TLR agonist selected from the group consisting of CpG oligonucleotides, flagellin, monophosphoryl lipid A, poly I:C, and derivatives thereof.

30. A method according to claim 29 wherein said adjuvant is poly I:C.

31. A method according to claim 17, wherein said adjuvant is a bacterial cell wall derivative selected from the group consisting of muramyl dipeptide (MDP), trehelose dycorynemycolate (TDM), and monophosphophoryl lipid A [MPL].

32. A method according to claim 16, further comprising administering the vaccine to the subject that has or is susceptible to a lymphoma or leukaemia.


SEQUENCE LISTING (6 sequences)

SEQ ID NO: 1 - DNA, 440 bp, Mus musculus
aagcttcaggacctcaccatgggatggagctggagctggatctttctctttctcctgtcaggaactgcaggtgtcctctctgaggttcagctacaacagtctggacctgacctggtgaagcctggggcttcagtgaagatatcctgcaagacttctggatacacattcactgaatacatcatgcactgggtgaagcagagccatggaaagagccttgagtggattggaggtattattcctaacaatggtggtactagctacaaccagaagttcaagttcaaggacaaggccacgatgactgtagacaagtcctccagcacaggttacatggaactccgcagcctgacatctgaggattctgcagtctattactgtacaaggcgagaggtgtacgggaggaattactatgctttggactactggggtcaaggaacactagt

SEQ ID NO: 2 - protein, 144 aa, Mus musculus
MGWSWSWIFLFLLSGTAGVLSEVQLQQSGPDLVKPGASVKISCKTSGYTFTEYIMHWVKQSHGKSLEWIGGIIPNNGGTSYNQKFKFKDKATMETTVDKSSSTGYMETELRSLTSEDSAVYYCTRREVYGRNYYALDYWGQGTL

SEQ ID NO: 3 - protein, 470 aa, artificial
  chimeric antibody comprising CD40 variable region fused to human IgG1 constant heavy chain
MGWSWIFLFLLSGTAGVLSEVQLQQSGPDLVKPGASVKISCKTSGYTFTEYIMHWVKQSHGKSLEWIGGIIPNNGGTSYNQKFKDKATMTVDKSSSTGYMELRSLTSEDSAVYYCTRREVYGRNYYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - DNA, 388 bp, Mus musculus
atgagggcccctgctcagttccttggtctcctgttgctctgttttcaaggtaccagatgtgatatccagatgacacagactacatcctccctgtctgcctctctgggagacagagtcaccatcacttgcagtgcaagtcagggcattaacaattatttaaactggtatcagcagaaaccagatggaactgttaaactcctgatctattacacatcaagtttacactcaggagtcccatcaaggttcagtggcagtgggtctgggacagattattctctcaccatcagcaacctggaacctgaagatattgccacttactattgtcagcagtatagtaaccttccgtacacgttcggaggggggaccaagctggaaataaaacgtacgg

SEQ ID NO: 5 - protein, 128 aa, Mus musculus
RAPAQFLGLLLLCFQGTRCDIQMTQTTSSLSASLGDRVTITCSASQGINNYLNWYQQKPDGTVKLLIYYTSSLHSGVPSRFSGSGSGTDYSLTISNLEPEDIATYYCQQYSNLPYTFGGGTKLEIKRT

SEQ ID NO: 6 - protein, 142 aa, Mus musculus
RAPAQFLGLLLLCFQGTRCDIQMTQTTSSLSASLGDRVTITCSASQGINNYLNWYQQKPDGTVKLLIYYTSSLHSGVPSRFSGSGSGTDYSLTISNLEPEDIATYYCQQYSNLPYTFGGGTKLEINVRLHHLSSSSRHLMSS